(19) United States
(12) Patent Application Publication
(10) Pub. No.: US 2026/0224177 A1
(43) Pub. Date: Aug. 6, 2026

(54) MEDICAL DEVICE ARRANGEMENT AND METHOD FOR DETECTING A DISTANCE SHORTFALL IN A MEDICAL DEVICE ARRANGEMENT

(71) Applicant: Siemens Healthineers AG, Forchheim (DE)

(72) Inventors: Verena SCHMIDT, Erbendorf (DE); Claudia IGNEY, Erlangen (DE); Chao ZHOU, Shanghai (CN)

(73) Assignee: Siemens Healthineers AG, Forchheim (DE)

(21) Appl. No.: 19/463,523

(22) Filed: Jan. 29, 2026

(30) Foreign Application Priority Data
Jan. 31, 2025 (DE) 10 2025 103 603.9

(51) Int. Cl. A61B 6/10; A61B 6/00; A61B 6/58; A61B 90/00

(52) U.S. Cl. CPC A61B 6/102; A61B 6/547; A61B 6/586; A61B 90/06

(57) ABSTRACT

A medical device arrangement includes a first movable device unit and a second movable device unit. The second movable device unit has a movable device arm with at least one hinged element and at least one sensor configure to capture the setting of the at least one hinged element. The medical device arrangement also includes a control unit with a signal link to the at least one sensor. A hinged setting signal captured by the sensor is transmitted to the control unit, and the control unit is configured to detect a distance shortfall between the first movable device unit and the second device unit based on the setting of the first movable device unit and the hinged setting signal.

### CROSS-REFERENCE TO RELATED APPLICATION(S)

The present application claims priority under 35 U.S.C. § 119 to German Patent Application No. 10 2025 103 603.9, filed Jan. 31, 2025, the entire contents of which is incorporated herein by reference.

### FIELD

One or more example embodiments of the present invention relate to a medical device arrangement as well as to a method for detecting distance shortfalls in a medical device arrangement.

### BACKGROUND

Independent of the grammatical term usage, individuals with male, female or other gender identities are included within the term.

Medical device arrangements frequently have a large number of individual device units which can move relative to one another. These include, for example, angiography systems which are frequently used for supporting operations by way of imaging. Systems of this kind can have an imaging apparatus, such as a CT device, MR device or, in particular, a C-arm device. Furthermore, angiography systems can have a screen or another kind of optical display, in particular so during an operation or treatment, a medical specialist can observe the medical images recorded with the imaging apparatus.

To enable a high degree of flexibility of use of the systems, in particular to enable situation-dependent positioning of the devices, the device units are designed so they can move relative to one another. During a treatment or operation, the imaging devices as well as a display for displaying the recorded medical images are moved around.

This entails the risk of the devices colliding with one another, and this can result in an interruption to the operation, damage to the devices and/or to failure of the medical device arrangement.

### SUMMARY

It is therefore an object of one or more example embodiments of the present invention to improve a medical device arrangement. In particular, one or more example embodiments of the present invention should make it possible to prevent damage to the medical device arrangement. In particular, one or more example embodiments of the present invention should make it possible to increase the availability of medical device arrangements. In particular, one or more example embodiments of the present invention should make it possible to facilitate operation of a medical device arrangement. In particular, one or more example embodiments of the present invention should make it possible to avoid collisions of device units of the medical device arrangement.

At least this object is inventively achieved by a method and an apparatus as claimed in the independent claims. Preferred embodiments are disclosed in the dependent claims.

According to one aspect of one or more example embodiments of the present invention, a medical device arrangement is provided. The medical device arrangement has a first movable device unit. The medical device arrangement also has a second movable device unit. The second movable device unit has a movable device arm. The device arm comprises at least one hinged element. At least one sensor is embodied for capturing the setting of the hinged element. Preferably, the sensor is arranged on the hinged element. The medical device arrangement also has at least one control unit with a signal link to the at least one sensor. A hinged setting signal captured by the sensor can be transmitted to the control unit. The control unit is embodied, on the basis of the hinged setting signal captured by the sensor and transferred to the control unit, to detect a distance shortfall between the first device unit and the second device unit. The control unit is preferably also embodied to detect a distance shortfall on the basis of the setting of the first device unit.

The term “movable device unit” should be understood broadly within the context of the present invention. In particular it should be taken to mean a device unit which can change its position and/or location in a space. For example, a device unit can be displaceable or swivelable. The following explanations generally refer to a device unit and thereby comprise the first as well as the second device unit.

The terms setting and/or position should be understood broadly in the present case. The terms are basically used synonymously in the present embodiments. They describe, in particular, where a body is situated relative to a coordinate system. In addition or alternatively, the terms describe the location or orientation of a body relative to the coordinate system. If reference is made in the present case to the capturing of a position or setting, for example, then capturing of where a body is situated and/or capturing of how the body is arranged is intended, with it being possible for only one of the two possibilities to exist or for both possibilities to be implemented.

Preferably, a device unit can be or comprise, for example, a medical imaging apparatus, which is movable in the space. The device unit can have, for example, a traversing and/or an adjusting system, in particular a rail system arranged on the floor, on a wall and/or on a ceiling, for example a rail system suspended from the ceiling. In addition or alternatively, the device unit can be embodied as what is known as a bi-planar system, which enables a movement in at least one, which is spanned by two axes. The device unit can additionally or alternatively be rotatable, so its location can be changed about a certain pivot point or an axis of rotation. In addition or alternatively, the device unit as a whole can be moved in the space, for example by a traversing system. The traversing system can have, for example, rollers by which the device unit can travel over the floor.

The device unit can have a movable, in particular swivelable, device arm. In the context of the present invention, swivelable can, in particular, be understood in such a way that one end of the device arm is fixed, for example on the ceiling of a space, and the device arm can move about this permanently installed end. In particular if the device unit comprises a display, it is possible for a first end of the device arm to be secured to a ceiling or it is secured to it. A second end of the device arm, opposite the first end, can comprise a display mount for securing a display. In addition or alternatively, an optical display can be secured to the second end.

The display can consequently be moved in the space by a movement of the device arm, in particular by a swiveling of the device arm. This has the advantage that a medical specialist can position the display so it is adapted to a specific application. For example, the position can be selected as a function of an action to be carried out during an operation. However, this simultaneously results in the risk of a collision of the display with other items or device units, such as an imaging apparatus, which it is possible, in particular, to also freely position. Of course, the first and/or the second device unit can comprise a device arm embodied within the meaning of the preceding embodiments.

The term “hinged element” should be understood broadly in the present case and comprises an element, which connects two component parts or one component part and a structure to one another and permits a relative movement of the component parts. For example, the hinged element can be used to connect two members of the device arm. However, a hinged element can also be provided for fastening a component part, for example of the device arm, to a structure in the surroundings, for example to a ceiling of an examination space. The hinged element can allow one or more types of relative movement. For example, the hinged element can enable a relative rotational movement of the connected component parts about an axis of rotation and/or enable a relative longitudinal movement of the connected component parts along a longitudinal axis.

In a preferred embodiment, the at least one hinged element is a swivel joint, that is to say it enables a rotation of a component part about a hinge axis or about a hinge point. The hinged element can be, for example, a hinge or a ball joint. The at least one sensor for capturing the setting of the swivel joint can be an angle sensor.

In a preferred embodiment, at least one hinged element is a rail element, that is to say the hinged element enables a longitudinal displacement of two component parts relative to one another along an axis. The at least one sensor for capturing the setting of the rail element can be displacement sensor. For example, the device arm can be secured to the ceiling of a space via a rail element.

The term “sensor” should be understood broadly within the context of the present disclosure and comprises, in particular, means, mechanisms and/or devices for capturing the setting of the at least one hinged element, irrespective of how the setting of the hinged element is specifically captured. It is merely significant that the setting is measured by the sensor. The sensors can supply, in particular, digital or analog output signals, which can be evaluated in an evaluation device, for example by an evaluation circuit.

The sensors can be embodied as incremental encoders or as absolute value encoders. Incremental encoders supply a specific number of incremental impulses per movement unit, with it being possible to ascertain a change in the setting on the basis thereof. For example, an optical division scale, a magnetic measurement or a toothed wheel or toothed rack arrangement can be used for capturing. The signal from an incremental encoder, together with a reference signal, which is characteristic of a zero setting or starting setting, can be used for position determination. Absolute value encoders supply an absolute measuring signal from which the position or setting of the joint can be directly derived.

Preferably, the setting of at least one part, in particular of one or more hinged element(s) of the device arm, can be measured, and/or the setting of one or more member(s) of the device arm can be measured. The setting of the device arm as a whole can be ascertained on the basis of the measurement of the setting of hinged elements and/or the setting of the members of the device arm. Alternatively or in addition, the setting of the device arm as a whole can also be measured, for example by a camera, to obtain a second position value, which can be used, for example, for verification purposes.

The sensor is preferably arranged on or in the hinged element. The sensor is thus protected against damage. In addition, the measurement of the setting of the hinged element is affected as little as possible by external influences. For example, a measurement of a sensor, which is arranged spaced apart from the hinged element, such as a camera capturing the setting of the joint arm, could be impeded or impaired by items or individuals situated between the sensor and the device arm. If, by contrast, the sensor is arranged on or in the hinged element, the measurement is affected less by disturbance variables. The accuracy of the measurement is consequently improved.

Preferably, the at least one sensor is therefore arranged on the at least one hinged element. Preferably, the at least one sensor is embodied for capturing the setting of the at least one hinged element. For example in the case of a swivel joint, a rotary encoder can be provided for capturing the relative movement of the arm members to one another. The setting captured by the rotary encoder can be transmitted to the control unit and thus be used for ascertaining the setting of the device arm. The position and/or the location of the second device in the space can be ascertained on the basis of the setting of the device arm.

The setting and/or position for a device unit can be compactly ascertained by arranging a sensor on the hinged element. In addition, the sensors are placed exactly where a relative movement occurs, namely in the joints, for which reason the measuring results are particularly accurate and are hardly affected by disturbance variables.

A signal link between the control unit and the sensor makes it possible for items of information to be transferred between the control unit and the sensor. The transferred items of information can comprise, in particular, measuring signals. For example, the measuring signals can be registered by the sensor and be transferred to the control unit for evaluation. However, it is also possible that the sensor comprises a decentral evaluation unit, which is embodied to evaluate the measuring signals registered by the sensor, in particular, to prepare the measuring signals for processing by the control unit.

The control unit can be embodied, for example, as an appropriately programmed data processing facility or said functionality can be implemented at least partially in the form of hardware. The control unit can be integrated in a device unit, in particular in medical imaging apparatus, or be embodied separately from it. It can be implemented, for example, as a workstation computer, server or Cloud solution. Furthermore, the control unit can be distributed among a plurality of different hardware components. For example, part of the control unit can be arranged in or on the first device unit and part of the control unit can be arranged in or on the second device unit.

The feature “detecting a distance shortfall” should be understood broadly in the present case. In particular, it can encompass that a distance shortfall of the first device unit, which actually occurs, is detected by the second device unit. In addition or alternatively, a distance shortfall occurring in the future can be detected, which would occur, for example, if a movement of the first and/or second device unit were to be executed as planned. This enables anticipatory control of the medical device arrangement, so potential distance shortfalls or other risks are detected as early as possible. In addition or alternatively, “detecting a distance shortfall” can also refer to distance shortfalls which took place in the past in order, for example, to simplify a subsequent analysis of distance shortfalls that have occurred.

A distance shortfall can be, in particular, a collision or near-collision of the first device unit with the second device unit. The control unit can be embodied to ascertain an instantaneous, a future and/or past distance between the first device unit and the second device unit. The ascertained distance can be compared with a distance limit value. When the distance limit value is undershot, a distance shortfall can be established by the control unit.

In a preferred embodiment, the control unit can be embodied to trigger an action when a distance shortfall is detected. For example, the control unit can be embodied to send a control signal to the first and/or second device unit. The control signal can be suitable for or embodied to induce a stoppage of the first device unit and/or the second device unit. The control signal can alternatively or additionally be suitable for and/or embodied to change a planned movement of the first device unit and/or second device unit. In addition or alternatively, the control unit can be embodied to trigger a warning signal when a distance shortfall is detected. A warning signal can be, in particular, an optical, acoustic, haptic or other kind of warning signal and/or can trigger or include an appropriate warning message. For example, an acoustic warning signal can be output and/or a warning message can be displayed on an optical display. In particular, a warning message can be displayed on an optical display of the second device unit. In addition or alternatively, a suggestion for an alternative movement and/or positioning of the first device unit and/or the second device unit can be output, in particular displayed.

Distance shortfalls between the first device unit and the second device unit can thereby be effectively detected by way of the construction described here. Detecting distance shortfalls, which have occurred or which may potentially occur, constitutes the first step for preventing distance shortfalls, and thus improving the safety and reliability of a medical device arrangement because, in particular, collisions of the first device unit with the second device unit are effectively stopped. Operation of the medical device arrangement is facilitated by the automatic position monitoring because it is no longer imperative for an operator to manually attend to adherence to a minimum distance between the first device unit and the second device unit. Damage to the device arrangement is avoided, whereby the availability of the device arrangement, that is to say the number of hours in which the device arrangement is ready for use, is increased.

In a preferred embodiment, the device arm is multi-membered, that is to say the device arm has a plurality of members. The device arm therefore has at least two members and a hinged element for connecting the two members of the device arm. More preferably, the device arm has a plurality of members, in particular two, three, four, five or a corresponding number of members, with one hinged element being arranged in each case between two adjacent arm members. A hinged element allows a movement of the arm members arranged close to one another relative to each other. For example, a swivel joint, which is regarded as a particular form of the hinged element, can permit a rotation of the one arm member relative to the other arm member about one or more joint axes.

Preferably, the first device unit has a position-capturing means or device with a signal link to the control unit. The position-capturing device can be embodied for capturing a current position, in particular a current location, a current movement, a planned position, in particular a planned location, and/or a planned movement of the first device unit. The position-capturing device can be, for example, a sensor for detecting the location and/or position and/or movement of the first device unit. In addition or alternatively, the location and/or position and/or movement of the first device unit can also be supplied to the control unit, for example, by a controller of the first device unit. In a particularly preferred embodiment, the control unit can comprise the controller of the first device unit.

A signal about the current position, the current movement, the planned position and/or the planned movement can consequently be transferred to the control unit and/or is available to the control unit. More preferably, the control unit is also embodied to detect a distance shortfall on the basis of the position, location and/or movement of the first device unit.

The planned or actual location, position and/or movement of the first device unit is thereby used in addition to the location and/or position of the device arm of the second device unit or the location and/or position of the second device unit itself for ascertaining the distance. The accuracy of ascertaining the distance shortfall is improved thereby. In addition, improved predictions about a future system behavior can thus be made, as will be stated in more detail below.

In a preferred embodiment, the first movable device unit comprises or is a medical imaging apparatus. In addition or alternatively, the second medical device unit can comprise an optical display arranged on the device arm. In this embodiment, for example, images recorded by the first device unit via the medical imaging apparatus are or can be displayed on the optical display of the second device unit. Preferably, the control unit is embodied to detect a distance shortfall between the first device unit and the device arm, in particular between the first device unit and the optical display, so damage to the optical display is avoided.

As already indicated above, the optical display is preferably arranged on one end of the device arm of the second device unit, so it can be positioned in the space by way of a movement, in particular a swivel movement, of the device arm. As a result, the optical display can be positioned particularly flexibly.

Preferably, the planned movement of the first device unit comprises a movement which is to be executed for a medical image recording. For example for the medical image recording, it may be necessary that the first device unit or an image-recording facility of the first device unit moves around a patient, which can be arranged, for example, on an operating table. Alternatively or in addition, the first device unit or an image-recording facility of the first device unit can move along a longitudinal axis, in particular a z-axis, for example in order to acquire a medical image of a certain volume range of a patient.

The movement of the image-recording facility necessary for recording one or more medical image(s) can be known to the control unit or can be transmitted to the control unit. The control unit can be embodied to calculate or ascertain a movement trajectory on the basis of precisely the planned movement of the imaging apparatus. The control unit can also be embodied to ascertain whether a distance from the second device unit is undershot by way of the ascertained movement trajectory and/or on execution of the planned movement of the imaging apparatus.

Hypothetically occurring collisions can thus be ascertained as early as in the planning stage of recording of a medical image, and the recording can be planned such that no infringement of a minimum distance between the first and the second device unit occurs during recording.

In some cases, various possible movements of the image-recording apparatus can be available for recording of a medical image. The control unit can be embodied, a planned movement of the imaging apparatus is modified in such a way that a medical image is recorded without undershooting the minimum distance between the first device unit and the second device unit.

In particular, an alternative movement can be proposed in this case for the recording of the same medical image. In addition or alternatively, the recording of a comparable image can be proposed, which is recorded via a movement of the imaging apparatus which does not result in a distance shortfall. The proposal for the alternative movement can be displayed and/or a corresponding control signal can be provided by the control unit, which signal prompts a corresponding alternative movement of the imaging apparatus.

Preferably, the second movable device unit can be swiveled by the device arm, in particular manually. A manual swivelability of the device arm has the advantage that no additional adjustment drives are necessary to swivel the device arm, whereby the device arm is particularly fail-safe.

Preferably, the control unit is embodied to detect a distance shortfall between the device arm of the second control unit and the first device unit. The control unit can therefore be embodied to trigger the movement of the device arm in a more refined manner, in particular by monitoring the hinged setting of the hinged elements of the device arm, in order to protect a specific part of the device arm, for example an end of the device arm or an item arranged on the end, against collisions or distance shortfalls by the first device unit.

In a preferred embodiment, on at least one of the hinged elements, the second movable device unit comprises an actuator with a signal link to the control unit. The setting of the hinged element can be adjusted by the actuator. Control signals can be transmitted from the control unit to the actuator in order to adjust the hinged setting.

Preferably, the distance shortfall can be detected on the basis of the control signals transmitted to the actuator and/or on the basis of the hinged setting adjusted by the actuator.

In addition or alternatively to ascertaining the setting of the device arm on the basis of the measuring signals of the at least one sensor, the setting of the device arm can consequently be ascertained or the ascertained setting of the device arm can be checked on the basis of the control signals sent to the actuators by the control unit. This ascertainment, based on the control signals which have been sent, can be used to check the setting ascertained on the basis of the measured values. Conversely, the control signals transmitted to the actuator can be checked by way of the hinged setting and/or arm setting ascertained on the basis of the measuring signals. Alternatively or in addition, the setting of the device arm can be ascertained on the basis of a combination of the measuring signals and the control signals. This has the advantage that reliable operation of the medical device arrangement is enabled. In addition, the position and setting of the device arm are regulated, in particular by the checking and monitoring of the measuring signals by way of the control signals, so the arm can be positioned more accurately and reliably.

In a preferred embodiment, the at least one sensor is simultaneously an actuator. For example, the sensor can be an actuating drive whose setting is captured by sensors. For example, a rotary encoder can be provided, which measures the setting of an electric motor and transmits a corresponding signal to the control unit. A combination of this kind of actuator and sensor has the advantage that the assembly is very compact and thus only a small installation space is required. However, it is not imperative that the setting of a joint element, which can be adjusted by an actuator, is captured by a sensor. As an alternative to the sensor, in such a case an actuator, such as an electric motor, can be provided whose control variables, for example an electrical current, can be captured by the control unit. The control unit can be embodied to ascertain a setting of the hinged element and/or of the entire device arm, and therewith of the second device unit, on the basis of the captured control variables.

For example, a servomotor can be used as an actuator, which allows monitoring of the hinged setting as well as, optionally, the actuating movement, that is to say the actuating speed and/or acceleration. Servomotors customarily have an electric motor and a sensor for position determination. Servomotors can be operated in a closed loop. Operation can be moment-, speed-or position-regulated. Combinations are possible due to the nesting of the closed loops. In the context of the present invention, the servomotor can simultaneously be used as a sensor and as an actuator due to the capture, by way of sensors, of the movement of the electric motor provided in the servomotor.

Alternatively or in addition, an electric step motor can be used as an actuator. Step motors can be accurately operated without a sensor system for position feedback and are thereby simpler and less expensive to construct than servomotors.

Preferably, the multi-jointed device arm can have a plurality of hinged elements. More preferably, a sensor for detecting the hinged setting is arranged on each of the hinged elements. In addition or alternatively, an actuator for altering the hinged setting can be arranged on each of the joints. Each of the sensors and/or actuators can have a signal link to the control unit. The setting of the device arm can thus be particularly accurately captured, controlled and/or regulated.

In a preferred embodiment, the control unit is embodied to ascertain the setting of the device arm using forward kinematics. In particular, the location of a suspension point of a first end of the device arm, for example on a ceiling of an operating room, can be known. Starting from this known point, the device arm can extend in a plurality of members up to its second end. Joints with one sensor respectively for capturing the hinged setting can be arranged between the members respectively. The measured values captured the sensors, which reflect the respective hinged setting, can be sent to the control unit. The control unit can have, for example in a memory of the control unit, access to items of information about the kinematic correlations, which apply to the device arm. In particular, it is possible via the kinematic correlations to describe how the setting of the device arm changes when one of the joints alters its setting. The control unit can be embodied to ascertain, on the basis of the kinematic correlations and on the basis of the measured values captured by the sensors, the position and/or location of the device arm in the space. In particular, the location of the second end of the device arm in the space can be ascertained. Thus, for example, the location of a display arranged on the second end of the device arm can be ascertained.

The setting of the device arm and/or the position of the second end of the device arm can be calculated in real time or quasi-real time, so even while the device arrangement is being used it is possible to monitor whether a distance shortfall exists. That the calculation is in real-time or quasi-real time means that the calculation lasts an acceptable period of time. An acceptable period of time can be, for example, less than 1 millisecond, less than 10 milliseconds, less than 100 milliseconds or less than 1 second.

In a preferred embodiment, when a distance shortfall between the first movable device unit and the second movable device unit is detected, the control unit is embodied to output a blocking signal, which causes a stoppage of movement of the first movable device unit and/or the second movable device unit. A collision of the first device unit with the second device unit can thus be reliably prevented and damage to the first device unit and/or second device unit can be avoided.

In a preferred embodiment, the control unit can be embodied to trigger a return movement of the first device unit and/or the second device unit if a distance shortfall between the first movable device unit and the second movable device unit is detected. Such a return movement can, for example, be directed away from the other device unit, so the return movement performs an evasive movement. A collision of the device units is thereby prevented even if one of the device units were to continue moving, for example owing to inertia. Safety during operation of a medical device arrangement is thereby increased further.

Preferably, when a distance shortfall of the first movable device unit is detected by the second movable device unit, the control unit can be embodied to propose an alternative position and/or alternative route for the movement of the first device unit and/or the second device unit. As already stated above, this results in it being possible for a function which is to be carried out by the first device unit or second device unit, for example the recording of a medical image, to be executed despite detection of a distance shortfall. The robustness of the functionality of the device arrangement is consequently improved.

The control unit can also be embodied to send a control signal to the first and/or second device unit, which causes the first and/or second device unit to be moved according to the ascertained or proposed alternative position and/or alternative route. The first and/or the second device unit, more precisely one or more actuator(s) of the corresponding device unit, can consequently be actuated by the control signal.

In a preferred embodiment, at least one inertial measuring unit (IMU) is arranged on the device arm of the second device unit. More preferably, an inertial measuring unit is arranged at least on the last member of the device arm, that is to say on the member on which the second end of the device arm is preferably embodied. More preferably, an inertial measuring unit is arranged on each member of the device arm. The inertial measuring unit is embodied to detect the location and/or the movement of the device arm and/or the member on which the inertial measuring unit is arranged. The at least one inertial measuring unit has a signal link to the control unit, with the control unit being embodied to check an ascertained position of the device arm and/or the second end of the device arm and/or the associated member of the device arm on the basis of the location and/or movement captured by the inertial measuring unit. The accuracy of the arm position ascertained by the control unit is thus increased. Furthermore, a monitoring possibility is created for the ascertained arm position.

The control unit can also be embodied to send a control signal to the second device unit, which causes the location and/or movement of the device arm to be adjusted when the checking establishes that the ascertained position of the device arm no longer conforms with the specified position of the device arm. The second device unit, more precisely one or more actuator(s) of the second device unit, can consequently be actuated accordingly by the control signal. The difference between a specified position and the position of the device arm which has actually been assumed can thus be reduced.

According to a further aspect, one or more example embodiments of the present invention relate to a method for detecting a distance shortfall in a medical device arrangement. The medical device arrangement can be embodied, in particular, according to one of the exemplary embodiments described above. The medical device arrangement at least has one first movable device unit and one second movable device unit with a movable device arm. The device arm has at least one hinged element and at least one sensor for capturing the setting of the at least one hinged element. The method has the following steps:
capturing the setting of the at least one hinged element;
ascertaining the setting of the movable device arm on the basis of the setting of the at least one hinged element;
detecting a distance shortfall between the first device unit and the second device unit on the basis of the setting of the first device unit and on the basis of the ascertained setting of the device arm of the second device unit.

Collisions between the first device unit and the second device unit, which could result in damage to the device units, are avoided by the method for detecting a distance shortfall in a medical device arrangement. In addition, the functionality and availability of a device arrangement is improved.

Features, which were discussed in the context of one of the exemplary embodiments relating to a device arrangement, can also be transferred with said advantages to the other disclosed subject matters of the present invention, in particular to the presented method for detecting a distance shortfall.

One or more example embodiments of the present invention also relate to a computer program with instructions which, when executed by a control unit, are configured to carry out the method for detecting a distance shortfall.

One or more example embodiments of the present invention also relate to a non-transitory computer-readable storage medium or data carrier with instructions which, when executed by a control unit, are configured to execute the method for detecting a distance shortfall.

### BRIEF DESCRIPTION OF THE DRAWINGS

Further advantages and details of the present invention can be found in the following exemplary embodiments as well as the associated drawings, in which schematically:

FIG. 1 shows an exemplary embodiment of a medical device arrangement, and

FIG. 2 shows a flowchart of a method for detecting a distance shortfall.

### DETAILED DESCRIPTION

The exemplary embodiments depicted in more detail below represent preferred embodiments of the present invention. To improve the readability of the following embodiments, identical reference numerals are used for mutually corresponding elements of comparable exemplary embodiments.

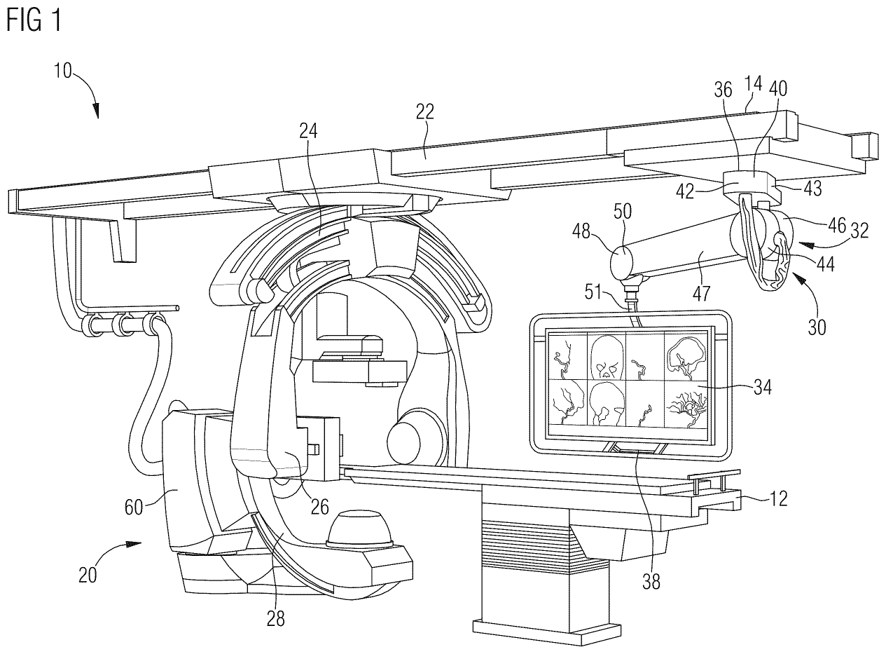

FIG. 1 shows schematically and in a simplified representation, a medical device arrangement 10, which can be used, for example, for X-ray imaging. The example of FIG. 1 represents a construction of a device arrangement 10 according to the principle of a C-arm device with an imaging apparatus 26, in particular a rotatable and movable C-arm 28, which can be rotated and moved accordingly in order to map an object and/or examination object which is to be mapped, such as by way of example a patient situated on a patient table 12, from different directions, i.e. with different recording angles.

The device arrangement 10 can be configured, for example, to carry out a rotational angiography method, for example on the basis of the principle of subtraction angiography. In this case, for example a large number of two-dimensional projections (also called output images here) can be generated from different angles. If necessary, a computing unit can calculate a three-dimensional reconstruction therefrom. However, a device arrangement 10 can also be constructed according to other constructions. In particular, the concept is, in principle, not limited to X-ray-based imaging methods.

In the present exemplary embodiment, the device arrangement 10 has a first device unit 20. The first device unit 20 comprises a traversing rail 22, along which an imaging apparatus 26 can be displaced. Apart from the C-arm 28 already mentioned, the imaging apparatus 26 comprises a rotation apparatus 24, by which the C-arm 28 can be rotated. Thus an examination object situated, in particular, on the patient table 12 can be recorded from different angles.

The movements of the C-arm 28 and the entire imaging apparatus 26 are controlled by a control unit 60. The imaging apparatus 26 and/or the C-arm 28 can be moved manually, that is to say by muscular strength and/or a manual movement specification. Alternatively or in addition, the imaging apparatus and/or the C-arm can be automatically moved, with the movement preferably being executed by control commands induced by the control unit 60.

The device arrangement 10 also has a second device unit 30. The second device unit 30 is mounted on a ceiling 14 of the space via a ceiling mount. The second device unit 30 has a swivelable, multi-jointed device arm 32 whose first end 36 is arranged on the ceiling mount. The first end 36 of the device arm 32 cannot thereby be moved relative to the ceiling. The device arm 32 has a large number of arm members 43, 47, 51, which are connected to one another via hinged elements 40, 44, 48 respectively. The hinged elements 40, 44, 48 are embodied as swivel joints.

A first arm member 43 is consequently fastened to the ceiling 14 by a first swivel joint 40, which defines an axis of rotation perpendicular to the ceiling 14, and can be swiveled in a plane parallel to the ceiling 14. A second arm member 47 is fastened to the first arm member 43 by a second hinged element 44, so the second arm member 47 can be swiveled about a joint axis relative to the first arm member 43. A third hinged element 48, which enables the third arm member 51 to swivel in respect of the second arm member 47, is arranged between the second arm member 47 and a third arm member 51. As a result, a second end 38 of the device arm 32 opposing the first end 36 can be moved in the space with a large degree of positioning freedom by swiveling the device arm 32. Arranged at the second end 38 is a screen 34, which can be positioned in the space by swiveling the device arm 32.

The free positioning of the screen 34 makes it possible for a user of the device arrangement 10 to orient the screen as a specific situation, for example during an operation, demands. However, this free positioning entails the risk of the screen 34, or the entire second device unit 30, colliding with the first device unit 20, in particular if the first device unit 20 and the second device unit 30 are positioned independently of one another respectively.

To avoid situations of this kind and, in particular, to ensure that a predefined distance between the first device unit 20 and the second device unit 30 is not undershot, sensors 42, 46, 50 are arranged on at least one, although in the present case on each, of the hinged elements 40, 44, 48, by which the setting of the hinged elements can be captured.

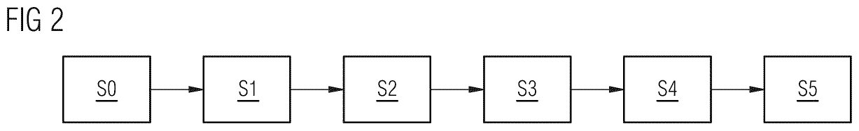

This enables the application of a method for detecting, and optionally avoiding, distance shortfalls between the first device unit 20 and the second device unit 30, as is schematically represented in FIG. 2 as a flowchart.

The method can be executed, in particular, in real time during use of the medical device arrangement.

In a step S1, the setting of the hinged elements 40, 44, 48 is ascertained by the sensors 42, 46, 50 and transmitted to the control unit 60 via a signal link. The device arm 32 of the second device unit 30 represented in FIG. 1 can be manually swiveled. However, the inventive idea also comprises that at least one of the hinged elements 40, 44, 48 is fitted with an actuator to be able to automatically adjust the setting of the corresponding hinged element 40, 44, 48.

In a step S2, the measuring signals captured by the sensors 42, 46, 50 are evaluated by the control unit 60. A setting of the device arm 32, in particular a positioning and/or orientation of the second end 38 of the device arm 32, is ascertained on the basis of the measuring signals. Items of information about the kinematics of the device arm 32, such as the lengths of the arm members 43, 47, 51 and the location and/or orientation of the joint axes can be used for this.

In a step S3, a positioning and/or orientation of the first device unit 20 is ascertained or obtained. The step S3 can preferably take place simultaneously with steps S1 and S2 but can also be executed at different times to these two steps. In particular, a next planned movement and/or action, which will be carried out by the first device unit 20, can be obtained and/or captured by the control unit 60.

In a step S4, on the basis of the positioning and/or the orientation of the first device unit 20 and the second device unit 30, the control unit 60 detects whether a collision or an undershooting of a minimum distance between the first device unit 20 and the second device unit 30 is taking place or will take place.

In a step S5, the control unit 60 prompts measures to avoid an undershooting of the minimum distance or prevent a collision. If an undershooting of the minimum distance can no longer be avoided or a collision can no longer be prevented, the measures prompted by the control unit 60 focus on reducing the damage which has been caused to a minimum. For example, the movement of the first and/or second device unit 20, 30 can be stopped by a control signal sent by the control unit 60. In addition or alternatively, an alternative route can be proposed for the movement of the first device unit 20 and/or the second device unit 30 and/or be triggered by corresponding control commands.

Optionally, the system can be calibrated in a preceding step S0. The first device unit 20 and/or the second device unit 30 can be brought into a predefined setting for this. In particular, the device arm 32 can be held in a predefined setting for the calibration and/or a predefined calibration trajectory can be covered.

The various illustrated logical blocks, modules, circuits and algorithm steps, which are described in connection with the disclosed exemplary embodiments, can be implemented as electronic hardware, computer software or combinations of the two. To clearly demonstrate this interchangeablity of hardware and software, various illustrative components, blocks, modules, circuits and steps were described in general above in relation to their functionality. Whether this functionality is implemented as hardware or software depends on the respective application, which are imposed on the overall system. Persons skilled in the art can implement the described functionality in different ways for each specific application, although such implementation decisions should not be interpreted as a deviation from the scope of this disclosure or the claims.

Embodiments realized as software can be implemented in software, firmware, middleware, microcode, hardware description languages or in any desired combination of these. A code segment or machine-readable instructions can represent a method, a function, a subprogram, a program, a routine, a subroutine, a module, a software packet, a class or any combination of instructions, data structures or program instructions. A code segment can be coupled to another code segment or a hardware circuit in that items of information, data, arguments, parameters or memory contents can be passed on and/or received. Items of information, arguments, parameters, data, etc. can be transmitted, routed or transferred, for example by memory release, message routing, token routing, network transmission, etc.

The actual software code or the specialized control hardware, which is used for implementing these systems and methods, is not limiting for the claimed features or this disclosure. Operation and behavior of the systems and methods were therefore described without reference to the specific software code, with it being understood that software and control hardware can be developed to implement the systems and methods on the basis of the description herein.

With the implementation as software, the functions can be stored as one or more instruction(s) or code on a non-volatile, computer-readable or processor-readable storage medium. The steps of a method or algorithm disclosed herein can be embodied in a software module executable by a processor, which can be located on a computer-readable or processor-readable storage medium. A non-volatile computer-readable or processor-readable storage medium comprises computer storage media as well as accessible storage media, which facilitate the transfer of a computer program from one location to another. A non-volatile processor-readable storage medium can be any available medium which a computer can access. For example, but not limiting, such non-volatile processor-readable media can comprise RAM, ROM, EEPROM, CD-ROM or other optical data carriers, magnetic data carriers or other magnetic data storage devices or any other accessible storage medium, which can store desired program code in the form of instructions or data structures and which a computer or processor can access. Disk/disc, as used here, comprise Compact Disk (CD), laser disk, optical disk, Digital Versatile Disk (DVD), floppy disk and Blu-ray disk, with disks usually reproducing data magnetically and disks reproducing data optically with lasers. Computer-readable media also include combinations of the above media. In addition, the operations of a method or algorithm can be in the form of a or any combination or series of codes and/or instructions on a non-volatile, processor-readable medium and/or computer-readable medium, which can be integrated in a computer program product.

The above description is intended to make it possible for a person skilled in the art to produce and/or use the exemplary embodiments described herein and variants thereof. Various modifications to these exemplary embodiments will be immediately obvious to persons skilled in the art, and the principles defined herein can be applied to other embodiments without deviating from the spirit or scope of the subject matter disclosed here. The present disclosure should therefore not be limited to the exemplary embodiments shown here, rather it should obtain the greatest possible scope according to the following claims and the principles disclosed herein and new features.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” OR “exemplary” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

While various aspects and exemplary embodiments were disclosed, other aspects and exemplary embodiments are also envisaged. The various disclosed aspects and exemplary embodiments are merely for the purpose of illustration and should not be regarded as limiting, with the true scope and spirit being defined by the following claims.

What is claimed is:

1. A medical device arrangement, comprising:
a first movable device unit;
a second movable device unit, wherein the second movable device unit includes a movable device arm with at least one hinged element and at least one sensor configured to capture a setting of the at least one hinged element; and
a control unit having a signal link to the at least one sensor for transmission of a hinged setting signal, captured by the at least one sensor, to the control unit,
wherein the control unit is configured to detect a distance shortfall between the first movable device unit and the second movable device unit based on a setting of the first movable device unit and the hinged setting signal.

2. The medical device arrangement as claimed in claim 1,
wherein the at least one hinged element includes a swivel joint, and the at least one sensor is an angle sensor configured to capture a setting of the swivel joint.

3. The medical device arrangement as claimed in claim 1,
wherein the at least one hinged element includes a rail element, and the at least one sensor includes a displacement sensor configured to capture a setting of the rail element.

4. The medical device arrangement as claimed in claim 1,
wherein
the first movable device unit has a position-capture device with a signal link to the control unit, the position-capture device configured to
capture at least one of a current position, a current movement, a planned position or a planned movement of the first movable device unit, and
transfer at least one of a position or movement of the first movable device unit to the control unit, and
the control unit is configured to detect the distance shortfall based on the at least one of the position or movement of the first movable device unit.

5. The medical device arrangement as claimed in claim 1,
wherein at least one of
the first movable device unit includes a medical imaging apparatus, or
the second movable device unit includes an optical display arranged on the movable device arm.

6. The medical device arrangement as claimed in claim 4,
wherein the planned movement of the first movable device unit includes a movement to be executed for a medical image recording.

7. The medical device arrangement as claimed in claim 1,
wherein the second movable device unit is configured to swivel, via the movable device arm, and wherein the control unit is configured to detect the distance shortfall between the movable device arm and the first movable device unit.

8. The medical device arrangement as claimed in claim 1,
wherein
on at least one of the at least one hinged element, the second movable device unit includes an actuator with a signal link to the control unit, by which the setting of the at least one hinged element is adjustable, and
for adjusting the setting of the at least one hinged element, control signals are transferred from the control unit to the actuator.

9. The medical device arrangement as claimed in claim 1,
wherein the at least one sensor is also an actuator.

10. The medical device arrangement as claimed in claim 1,
wherein
the movable device arm has a plurality of hinged elements, arranged on each of the plurality of hinged elements is at least one of
a sensor configured to detect the setting of the at least one hinged element, or
an actuator configured to alter the setting of the at least one hinged element, and
at least one of each sensor or each actuator has a signal link to the control unit.

11. The medical device arrangement as claimed in claim 1,
wherein the control unit is configured to ascertain a setting of the movable device arm using forward kinematics.

12. The medical device arrangement as claimed in claim 1,
wherein when the distance shortfall of the first movable device unit is detected by the second movable device unit, the control unit is configured to output a blocking signal, which causes a stoppage of movement of at least one of the first movable device unit or the second movable device unit.

13. The medical device arrangement as claimed in claim 1,
wherein when the distance shortfall between the first movable device unit and the second movable device unit is detected, the control unit is configured to propose at least one of an alternative position or alternative route for movement of at least one of the first movable device unit or the second movable device unit.

14. The medical device arrangement as claimed in claim 1, further comprising:
at least one inertial measuring unit arranged on the movable device arm of the second movable device unit, the at least one inertial measuring unit having a signal link to the control unit and being configured to detect at least one of a location or movement of the movable device arm, wherein
the control unit is configured to check an ascertained position of the movable device arm based on the at least one of the location or movement captured by the at least one inertial measuring unit.

15. A method for detecting a distance shortfall in a medical device arrangement, wherein the medical device arrangement includes a first movable device unit and a second movable device unit with a movable device arm, wherein the movable device arm has at least one hinged element and at least one sensor configured to capture a setting of the at least one hinged element, and wherein the method comprises:
capturing the setting of the at least one hinged element;
ascertaining a setting of the movable device arm based on the setting of the at least one hinged element; and
detecting a distance shortfall between the first movable device unit and the second movable device unit based on a setting of the first movable device unit and the setting of the movable device arm of the second movable device unit.

16. The medical device arrangement as claimed in claim 7, wherein the second movable device unit is configured to swivel manually via the movable device arm.

17. The medical device arrangement as claimed in claim 8,
wherein the distance shortfall is detected based on at least one of the control signals transferred to the actuator or the setting of the at least one hinged element adjusted by the actuator.

18. The medical device arrangement as claimed in claim 4,
wherein at least one of
the first movable device unit includes a medical imaging apparatus, or
the second movable device unit includes an optical display arranged on the movable device arm.

19. The medical device arrangement as claimed in claim 4,
wherein the second movable device unit is configured to swivel, via the movable device arm, and wherein the control unit is configured to detect the distance shortfall between the movable device arm and the first movable device unit.

20. The medical device arrangement as claimed in claim 4,
wherein
on at least one of the at least one hinged element, the second movable device unit includes an actuator with a signal link to the control unit, by which the setting of the at least one hinged element is adjustable, and
for adjusting the setting of the at least one hinged element, control signals are transferred from the control unit to the actuator.
